(19) United States
(12) Patent Application Publication
(10) Pub. No.: US 2017/0281587 A1
(43) Pub. Date: Oct. 5, 2017

(54) ERYTHROCYTE FUNCTION IMPROVING AGENT

(71) Applicant: Glico Nutrition Co., Ltd., Osaka-shi, Osaka (JP)

(72) Inventors: Azusa NISHINO, Osaka-shi, Osaka (JP); Takashi ICHIHARA, Osaka-shi, Osaka (JP)

(73) Assignee: Glico Nutrition Co., Ltd., Osaka-shi, Osaka (JP)

(21) Appl. No.: 15/507,736

(22) PCT Filed: Sep. 1, 2015
(86) PCT No.: PCT/JP2015/074856

§ 371 Date: Feb. 28, 2017

(30) Foreign Application Priority Data
Sep. 1, 2014 (JP) 2014-177118

(51) Int. Cl. A61K 31/34; A61K 9/00

(52) U.S. Cl. CPC A61K 31/34; A61K 9/0053

(57) ABSTRACT

An object of the present invention is to provide an agent for improving red blood cell function which has both high antioxidant action and high ability to migrate into red blood cells, and can effectively protect red blood cells against oxidative stress. Cucurbitaxanthin A and/or a derivative thereof has an ability to migrate into red blood cells significantly higher than that of the other carotenoids reported to migrate into red blood cells, and can effectively protect red blood cells against oxidative stress, and thus, is effective as an agent for improving red blood cell function.

### TECHNICAL FIELD

The present invention relates to an agent for improving red blood cell function. More specifically, the present invention relates to an agent for improving red blood cell function which is effective in protecting red blood cells against oxidative stress, and improving the oxygen-carrying capacity.

### BACKGROUND ART

Red blood cells serve to carry oxygen to the tissues of the whole body, and play a very important function in maintaining life. On the other hand, red blood cells are constantly exposed to oxidative stress in blood due to reactive oxygen species and the like, and are in an environment where they are readily subjected to oxidative damage. Red blood cells inherently have such flexibility that they are freely deformable, and can change their shape and flow in blood capillaries smaller than red blood cells. If, however, the oxidative stress of red blood cells accelerates and their membrane lipids are oxidized, the red blood cells become hardened and cannot readily flow in small blood vessels, leading to a decrease in oxygen-carrying capacity. Further, if the acceleration of the oxidative stress of red blood cells causes oxidation of hemoglobin, conversion to methemoglobin lacking an oxygen-binding capacity occurs, also leading to a decrease in oxygen-carrying capacity (arterial blood oxygen saturation). It is known that such a decrease in the oxygen-carrying capacity of red blood cells causes a decrease in physical function and acceleration of aging, and is also responsible for diseases such as Alzheimer's disease, arteriosclerosis, hypertension, diabetes, hyperlipidemia, cataract, and apoplexy. It is thus important to protect blood cells against oxidative stress, and improve the function of red blood cells, in order to enhance physical function, and prevent or treat diseases caused in part by a decrease in the function of red blood cells.

Carotenoids are naturally occurring pigment components that are widely distributed in plants and animals, and there are numerous types of carotenoids, reportedly over 700. Carotenoids have a basic structure including a polyene portion composed of nine conjugated double bonds and end groups attached to both ends thereof. These carotenoids, in general, are considered beneficial to human health, which is the reason why the intake of brightly colored vegetables is recommended. The health function of the carotenoids is broadly classified into the known functions, i.e., the provitamin A function and the antioxidant function.

The provitamin A function refers to the conversion of some of ingested carotenoids into vitamin A within a human body. Vitamin A is an essential component for the light perception mechanism in the retina, and vitamin A deficiency leads to night blindness. Only a few carotenoids having the provitamin A function are known, such as β-carotene and β-cryptoxanthin, and this function is absent in many carotenoids (such as astaxanthin, lutein, zeaxanthin, capsanthin, capsorubin, and cucurbitaxanthin A). It is known that the carotenoids having the provitamin A function, when absorbed into the body, are actively taken up by the liver, where they are converted into vitamin A, as required, and migrate into the retina.

On the other hand, the antioxidant function is the function possessed by all the carotenoids. Active oxygen, which is constantly produced in the tissues, cells, intracellular organelles, and the like within the body, is believed to be one cause of lifestyle-related diseases, cancer, arteriosclerosis, and the like. Substances having antioxidant activity such as carotenoids are believed to be involved in health maintenance, by scavenging active oxygen produced within the body. However, not all the carotenoids absorbed into the body demonstrate the antioxidant function. The intensity of the antioxidant activity and the biokinetics (the property of migrating into tissues or cells) of each of the carotenoids are very important factors for the demonstration of the antioxidant function.

Conventionally, studies examining correlations between the structures of various carotenoids and their antioxidant action have shown that the intensity of the antioxidant action of carotenoids is largely dependent on the type of the carotenoid, in particular, the structure of terminal end groups. Specifically, Non Patent Literature 1 has reported that carotenoids having 3,6-epoxy end groups or 5-membered ring end groups (x-end groups) have high antioxidant action, and these carotenoids exhibit improved antioxidant action (singlet oxygen scavenging action) compared to carotenoids without these end groups.

Representative examples of carotenoids having 3,6-epoxy end groups or 5-membered ring end groups include capsanthin, capsorubin, cucurbitaxanthin A, and capsanthin 3,6-epoxide, and representative examples of carotenoids without these end groups include β-carotene, zeaxanthin, and lutein. FIG. 1 shows the structures of carotenoids having 3,6-epoxy end groups or 5-membered ring end groups, as well as the structure of a carotenoid without these end groups. It is known that carotenoids having 3,6-epoxy end groups or 5-membered ring end groups are localized in specific plant species, rather than being widely distributed in plants in general. For example, plants of the genus Capsicum such as paprika are known to contain carotenoids having 3,6-epoxy end groups and carotenoids having 5-membered ring end groups. Plants of the genus Cucurbitaceae such as pumpkins are also known to contain carotenoids having 3,6-epoxy end groups.

On the other hand, it has been shown that the biokinetics of carotenoids absorbed through the intestinal tract vary greatly depending on the type of the carotenoid. For example, it is known that β-carotene and β-cryptoxanthin are readily taken up by the liver, and lutein and zeaxanthin are localized in the tissue called the macula of the eye. It is also known that astaxanthin, which is localized in salmon eggs, crab shells, and the like, is excreted in humans without being taken up in organs or tissues.

It has been reported recently that some carotenoids contribute to improvement of the function of red blood cells by migrating into red blood cells after being orally ingested, and there are growing expectations that this will serve as a new health function of carotenoids.

Non Patent Literature 2, for example, compares carotenoids present in red blood cells between Alzheimer patients and their spouses. Non Patent Literature 2 has revealed that the major carotenoids present in the red blood cells of healthy individuals are lutein, zeaxanthin, β-carotene, and β-cryptoxanthin, whereas the red blood cells of Alzheimer patients show a decrease in lutein, zeaxanthin, and β-cryptoxanthin, along with an increase in lipid peroxides, and has reported the possibility that the decrease in these three xanthophylls (in particular, lutein) may reduce the function of red blood cells, and be one cause of the Alzheimer's disease.

Non Patent Literature 3 has reported that as a result of the examination of carotenoids in the red blood cells of 12 healthy males and females who orally ingested chlorella having a high lutein content for 4 weeks, an increase in lutein along with a decrease in lipid peroxides in the red blood cells were observed. These results indicate that the oral ingestion of carotenoids (specifically lutein) capable of migrating into red blood cells is effective for improving red blood cell function. Lutein, however, does not have a 3,6-epoxy end group or 5-membered ring end group at the ends, and cannot exhibit particularly high antioxidant action. Non Patent Literature 3 also discloses that red blood cells contain carotenoids other than lutein, such as zeaxanthin, β-carotene, and β-cryptoxanthin; however, these carotenoids also do not have 3,6-epoxy end groups or 5-membered ring end groups at the ends, and cannot exhibit particularly high antioxidant action.

Non Patent Literature 4 has reported that as a result of the examination of carotenoids in the red blood cells of 30 healthy males and females who received the oral administration of astaxanthin for 12 weeks, an increase in astaxanthin along with a decrease in lipid peroxides in the red blood cells were observed. Further, Patent Literature 1 has reported that astaxanthin or an ester thereof has the action of suppressing oxidative damage to red blood cells, preventing hardening of red blood cells, and stabilizing red blood cells, for example. Astaxanthin has very high antioxidant action although it does not have a 3,6-epoxy end group or 5-membered ring end group at the ends. From the disclosure of Non Patent Literature 3, however, the migration ratio of astaxanthin into red blood cells can be calculated as 0.0089% or 0.0057% on day 84 after the ingestion, and thus, astaxanthin has the drawback of having a very low ability to migrate into red blood cells.

Thus, if it is possible to find a component having markedly high antioxidant action and capable of effectively migrating into red blood cells, the function of red blood cells can be expected to be more effectively improved. Non Patent Literature 5, however, discloses that carotenoids having epoxy end groups have not been found in human blood, and are not absorbed. From the conventional art, therefore, it is currently believed that carotenoids having 3,6-epoxy end groups that exhibit particularly high antioxidant action do not have the ability to migrate into red blood cells.

### CITATION LIST

### Non Patent Literature

Non Patent Literature 1: Takashi Maoka et al., J. Oleo Sci., 50(8), 663-665 (2001)
Non Patent Literature 2: Takehiro kiko et al., J. Alzheimers Dis., 28(3), 593-600 (2012)
Non Patent Literature 3: Taiki Miyazawa et al., J. Oleo Sci., 62(11), 873-881 (2013)
Non Patent Literature 4: Kiyotaka Nakagawa et al., British Journal of Nutrition, 105,1563-1571 (2011)
Non Patent Literature 5: Takashi Maoka, Food and Clinical Nutrition, 2, 3-14, 2007

### Patent Literature

Patent Literature 1: Japanese Unexamined Patent Publication No. 2002-226368

### SUMMARY OF INVENTION

### Technical Problem

An object of the present invention is to provide an agent for improving red blood cell function which has both high antioxidant action and high ability to migrate into red blood cells, and can effectively protect red blood cells against oxidative stress.

### Solution to Problem

The present inventor conducted extensive research to solve the aforementioned problem, and found that, of the carotenoids having 3,6-epoxy end groups or 5-membered ring end groups which possess high antioxidant activity, only cucurbitaxanthin A having 3,6-epoxy end groups and/or a derivative thereof has a significantly high ability to migrate into red blood cells, and cucurbitaxanthin A and/or a derivative thereof can effectively protect red blood cells against oxidative stress, and is effective as an agent for improving red blood cell function. The present invention was completed as a result of further continued research based on this finding.

In summary, the present invention provides the following aspects of invention.

Item 1: An agent for improving red blood cell function comprising cucurbitaxanthin A and/or a derivative thereof as an active ingredient.

Item 2. The agent for improving red blood cell function according to item 1, wherein cucurbitaxanthin A and/or a derivative thereof is derived from paprika.

Item 3. The agent for improving red blood cell function according to item 1 or 2, which is an additive for a food or beverage product.

Item 4. A pharmaceutical preparation for oral administration for use in improving red blood cell function, comprising the agent for improving red blood cell function according to item 1 or 2.

Item 5. An agent for protecting red blood cells against oxidative stress comprising cucurbitaxanthin A and/or a derivative thereof as an active ingredient.

Item 6. An agent for improving oxygen-carrying capacity of red blood cells comprising cucurbitaxanthin A and/or a derivative thereof as an active ingredient.

Item 7. An agent for suppressing oxidative damage to red blood cells comprising cucurbitaxanthin A and/or a derivative thereof as an active ingredient.

Item 8. An agent for improving physical function comprising cucurbitaxanthin A and/or a derivative thereof as an active ingredient.

Item 9. An agent for recovering physical function during or after exercise comprising cucurbitaxanthin A and/or a derivative thereof as an active ingredient.

Item 10. An agent for improving cardiopulmonary function comprising cucurbitaxanthin A and/or a derivative thereof as an active ingredient.

Item 11. An agent for maintaining or improving brain function comprising cucurbitaxanthin A and/or a derivative thereof as an active ingredient.

Item 12. An agent for preventing or treating dementia comprising cucurbitaxanthin A and/or a derivative thereof as an active ingredient.

Item 13. An agent for preventing or treating a disease caused in part by a decrease in oxygen-carrying capacity of red blood cells, comprising cucurbitaxanthin A and/or a derivative thereof as an active ingredient.

Item 14. Use of cucurbitaxanthin A and/or a derivative thereof for the manufacture of an agent for improving red blood cell function.

Item 15. Use of cucurbitaxanthin A and/or a derivative thereof for the manufacture of an agent for protecting red blood cells against oxidative stress.

Item 16. Use of cucurbitaxanthin A and/or a derivative thereof for the manufacture of an agent for improving oxygen-carrying capacity of red blood cells.

Item 17. Use of cucurbitaxanthin A and/or a derivative thereof for the manufacture of an agent for suppressing oxidative damage to red blood cells.

Item 18. Use of cucurbitaxanthin A and/or a derivative thereof for the manufacture of an agent for improving physical function.

Item 19. Use of cucurbitaxanthin A and/or a derivative thereof for the manufacture of an agent for recovering physical function during or after exercise.

Item 20. Use of cucurbitaxanthin A and/or a derivative thereof for the manufacture of an agent for improving cardiopulmonary function.

Item 21. Use of cucurbitaxanthin A and/or a derivative thereof for the manufacture of an agent for maintaining or improving brain function.

Item 22. Use of cucurbitaxanthin A and/or a derivative thereof for the manufacture of an agent for preventing or treating dementia.

Item 23. Use of cucurbitaxanthin A and/or a derivative thereof for the manufacture of an agent for preventing or treating a disease caused in part by a decrease in oxygen-carrying capacity of red blood cells.

Item 24. Cucurbitaxanthin A and/or a derivative thereof used in a treatment for improving red blood cell function.

Item 25. Cucurbitaxanthin A and/or a derivative thereof used in a treatment for protecting red blood cells against oxidative stress.

Item 26. Cucurbitaxanthin A and/or a derivative thereof used in a treatment for improving oxygen-carrying capacity of red blood cells.

Item 27. Cucurbitaxanthin A and/or a derivative thereof used in a treatment for suppressing oxidative damage to red blood cells.

Item 28. Cucurbitaxanthin A and/or a derivative thereof used in a treatment for improving physical function.

Item 29. Cucurbitaxanthin A and/or a derivative thereof used in a treatment for recovering physical function during or after exercise.

Item 30. Cucurbitaxanthin A and/or a derivative thereof used in a treatment for improving cardiopulmonary function.

Item 31. Cucurbitaxanthin A and/or a derivative thereof used in a treatment of recovery for maintaining or improving brain function.

Item 32. Cucurbitaxanthin A and/or a derivative thereof used in a treatment of recovery for preventing or treating dementia.

Item 33. Cucurbitaxanthin A and/or a derivative thereof used in a treatment for preventing or treating a disease caused in part by a decrease in oxygen-carrying capacity of red blood cells.

Item 34. A method of improving red blood cell function comprising the step of administering, to an individual in need of improvement of red blood cell function, cucurbitaxanthin A and/or a derivative thereof in an amount effective for improving red blood cell function.

Item 35. A method of protecting red blood cells against oxidative stress comprising the step of administering, to an individual in need of protection of red blood cells against oxidative stress, cucurbitaxanthin A and/or a derivative thereof in an amount effective for protecting red blood cells against oxidative stress.

Item 36. A method of improving oxygen-carrying capacity of red blood cells comprising the step of administering, to an individual in need of improvement of oxygen-carrying capacity of red blood cells, cucurbitaxanthin A and/or a derivative thereof in an amount effective for improving oxygen-carrying capacity of red blood cells.

Item 37. A method of suppressing oxidative damage to red blood cells comprising the step of administering, to an individual in need of suppression of oxidative damage to red blood cells, cucurbitaxanthin A and/or a derivative thereof in an amount effective for suppressing oxidative damage to red blood cells.

Item 38. A method of improving physical function comprising the step of administering, to an individual in need of improvement of physical function, cucurbitaxanthin A and/or a derivative thereof in an amount effective for improving physical function.

Item 39. A method of recovering physical function during or after exercise comprising the step of administering, to an individual in need of recovery of physical function during or after exercise, cucurbitaxanthin A and/or a derivative thereof in an amount effective for recovering physical function.

Item 40. A method of improving cardiopulmonary function comprising the step of administering, to an individual in need of improvement of cardiopulmonary function, cucurbitaxanthin A and/or a derivative thereof in an amount effective for improving cardiopulmonary function.

Item 41. A method of maintaining or improving brain function comprising the step of administering, to an individual in need of maintenance or improvement of brain function, cucurbitaxanthin A and/or a derivative thereof in an amount effective for maintaining or improving brain function.

Item 42. A method of preventing or treating dementia comprising the step of administering, to an individual in need of prevention or treatment of dementia, cucurbitaxanthin A and/or a derivative thereof in an amount effective for preventing or treating dementia.

Item 43. A method of preventing or treating a disease caused in part by a decrease in oxygen-carrying capacity of red blood cells, comprising the step of administering a therapeutically effective amount of cucurbitaxanthin A and/or a derivative thereof to a patient having a disease caused in part by a decrease in oxygen-carrying capacity of red blood cells or an individual in need of prevention of the disease.

### Advantageous Effects of Invention

The agent for improving red blood cell function of the present invention can exhibit the protective action against oxidative stress of red blood cells, and improve the function of red blood cells, through efficient migration of cucurbitaxanthin A and/or a derivative thereof having markedly high antioxidant action into red blood cells. The agent for improving red blood cell function of the present invention is also effective for improving physical function, in particular, recovering physical function during or after exercise or improving cardiopulmonary function. The agent for improving red blood cell function of the present invention is also effective for maintaining or improving brain function, which requires a large amount of oxygen, because the agent for improving red blood cell function can improve the oxygen-carrying capacity through improvement of red blood cell function. The agent for improving red blood cell function of the present invention is also effective for preventing or treating a disease caused in part by a decrease in oxygen-carrying capacity of red blood cells, such as dementia, because the agent for improving red blood cell function can improve the oxygen-carrying capacity through improvement of red blood cell function.

Further, the agent for improving red blood cell function of the present invention can ensure high safety, because cucurbitaxanthin A and/or a derivative thereof, which has been eaten for a long time in the past, is used as an active ingredient.

### BRIEF DESCRIPTION OF DRAWINGS

FIG. 1 shows exemplary structures of carotenoids having 3,6-epoxy end groups or 5-membered ring end groups, as well as a carotenoid without these end groups.

FIG. 2 is a bar graph showing the total amount of each of the carotenoids ingested over 4 weeks in Example 1.

FIG. 3 is a bar graph showing the results of measurement of the migration ratio (%) into red blood cells of the amount of each of the carotenoids in Example 1.

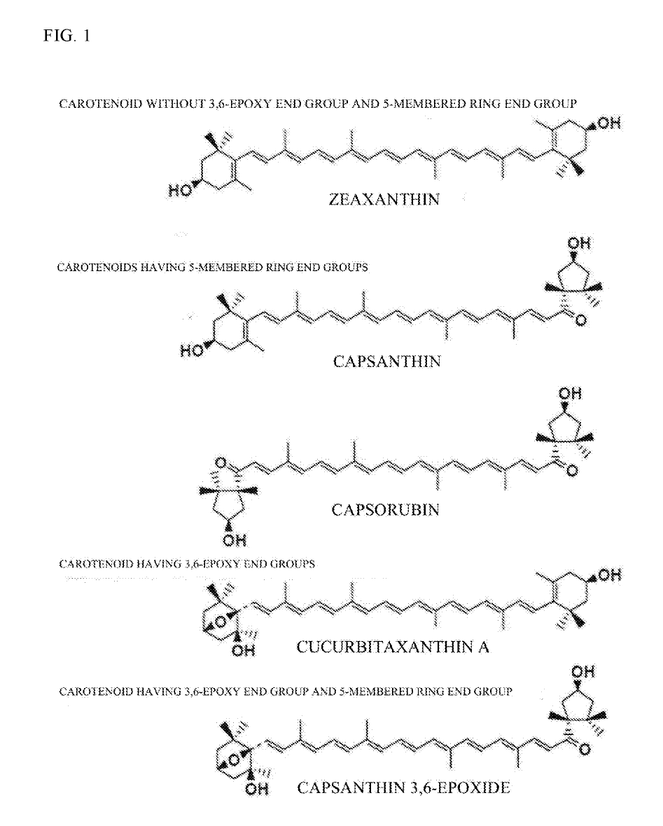

### DESCRIPTION OF EMBODIMENTS

The agent for improving red blood cell function of the present invention contains cucurbitaxanthin A and/or a derivative thereof as an active ingredient. The agent for improving red blood cell function of the present invention will be hereinafter described.

Active Ingredient

In the agent for improving red blood cell function of the present invention, cucurbitaxanthin A and/or a derivative thereof is used as an active ingredient.

Cucurbitaxanthin A, which has 3,6-epoxy end groups at the ends, is a carotenoid having antioxidant action, also referred to as (3S,3′R,5R,6R)-3,6-epoxy-5,6-dihydro-β,β-carotene-3′,5-diol.

Specific examples of derivatives of cucurbitaxanthin A include fatty acid esters of cucurbitaxanthin A. Specific examples of fatty acid esters of cucurbitaxanthin A include esters of saturated or saturated fatty acids containing 12 to 22 carbon atoms, such as lauric acid, myristic acid, palmitic acid, stearic acid, and oleic acid.

It is known that cucurbitaxanthin A and/or a derivative thereof is contained in natural products, for example, plants of the genus Capsicum such as paprika and plants of the genus Cucurbitaceae such as pumpkins. In the present invention, it is preferred to use cucurbitaxanthin A and/or a derivative thereof extracted from these natural products; however, cucurbitaxanthin A and/or a derivative thereof chemically or enzymatically synthesized or semi-synthesized may also be used.

In the agent for improving red blood cell function of the present invention, either one of cucurbitaxanthin A and a derivative thereof may be used alone, or a combination thereof may be used, as the active ingredient; however, a preferred example is cucurbitaxanthin A.

Cucurbitaxanthin A and/or a derivative thereof used in the present invention may not necessarily be a purified product, and may be a mixture containing carotenoids other than cucurbitaxanthin A and/or a derivative thereof. For example, paprika oleoresin derived from paprika contains a high concentration of cucurbitaxanthin A, and therefore, paprika oleoresin can be used as the active ingredient in the present invention.

The Dose of Cucurbitaxanthin A and/or a Derivative Thereof

The dose of the agent for improving red blood cell function of the present invention may be an amount effective for improving red blood cell function, and may be set as appropriate in accordance with the type or form, the use, the expected effects, the mode of administration, and the like of the product to be used. For example, the daily amount of ingestion or administration of cucurbitaxanthin A and/or a derivative thereof for an adult may be set to 0.001 to 20 mg, preferably 0.002 to 18 mg, and more preferably 0.005 to 15 mg.

Use

The agent for improving red blood cell function of the present invention is used for the purpose of improving the function of red blood cells, because the agent for improving red blood cell function can exhibit the protective action against oxidative stress of red blood cells, and improve the function of red blood cells, through efficient migration of cucurbitaxanthin A and/or a derivative thereof into red blood cells.

The protective action against oxidative stress of red blood cells is also effective for improving the oxygen-carrying capacity of red blood cells through improvement of arterial blood oxygen saturation, suppressing oxidative damage to red blood cells, and stabilizing red blood cells, for example. Thus, the agent for improving red blood cell function of the present invention can also be used as an agent for protecting red blood cells against oxidative stress, an agent for improving oxygen-carrying capacity of red blood cells, an agent for suppressing oxidative damage to red blood cells, and an agent for stabilizing red blood cells, for example.

Further, the improvement of the oxygen-carrying capacity of red blood cells is effective for improving physical function, and is particularly also effective for recovering physical function during or after exercise. Thus, the agent for improving red blood cell function of the present invention can also be used as an agent for improving physical function, an agent for improving cardiopulmonary function, and an agent for recovering physical function during or after exercise, for example. Further, the improvement of the oxygen-carrying capacity of red blood cells is also effective for maintaining or improving the function of brain, which is a tissue that requires a large amount of oxygen. Thus, the agent for improving red blood cell function of the present invention can also be used as an agent for maintaining or improving brain function.

The improvement of the oxygen-carrying capacity of red blood cells is also effective for preventing or treating a disease caused in part by a decrease in oxygen-carrying capacity of red blood cells. Thus, the agent for improving red blood cell function of the present invention can also be used as an agent for preventing or treating a disease caused in part by a decrease in oxygen-carrying capacity of red blood cells. Specific examples of diseases caused in part by a decrease in oxygen-carrying capacity of red blood cells include dementia such as Alzheimer-type dementia, arteriosclerosis, hypertension, diabetes, hyperlipidemia, cataract, and apoplexy.

Forms of Use of the Agent for Improving Red Blood Cell Function

There is no particular limitation to the mode of administration of the agent for improving red blood cell function of the present invention, as long as it is absorbed into a living organism, and examples of modes of administration include oral, enteral, transvenous, transarterial, subcutaneous, and intramuscular administration. Of these modes of administration, a preferred example is oral administration, from the standpoint of causing cucurbitaxanthin A and/or a derivative thereof to be easily and efficiently migrated into red blood cells.

The agent for improving red blood cell function of the present invention is incorporated into a product that needs to be provided with the action of improving the function of red blood cells. There is no particular limitation to the product into which the agent for improving red blood cell function of the present invention can be incorporated, and examples of products include food or beverage products and pharmaceutical preparations.

The dosage form of the product into which the agent for improving red blood cell function of the present invention is incorporated may be any of solid, semi-solid, liquid, and like dosage forms, and is set as appropriate in accordance with the type or use of the product. The product into which the agent for improving red blood cell function of the present invention is incorporated may contain food raw materials, food additives, nutritional components, pharmacologically acceptable bases, pharmacologically acceptable additives, pharmacological components, and the like, within a range where the effects of the present invention are not impaired. The product into which the agent for improving red blood cell function of the present invention is incorporated may also contain components that can improve the function of red blood cells, other than the agent for improving red blood cell function of the present invention. Examples of such components include lutein, zeaxanthin, β-cryptoxanthin, and astaxanthin.

When the agent for improving red blood cell function of the present invention is used in the field of food or beverage products, cucurbitaxanthin A and/or a derivative thereof as is or in combination with other food raw materials or additive components may be prepared into a desired form, and provided as a food or beverage product that achieves the effect of improving red blood cell function. Examples of such food or beverage products include, in addition to general food or beverage products, foods for specified health uses, nutritional supplements, functional foods, and foods for sick people. Specific examples of forms of these food or beverage products include, but are not particularly limited to, main dishes such as bread and noodles; side dishes such as cheese, ham, vienna sausages, and processed seafood products; confectionery such as gums, chocolates, soft candies, hard candies, biscuits, cookies, crackers, deep-fried rice crackers (“okaki” in Japanese), rice crackers (“senbei” in Japanese), and puffed snacks; chilled deserts such as ice creams, soft serve ice creams, sorbets, and frozen desserts; supplements such as tablets, granules, powders, capsules, and soft capsules; and beverages such as soft drinks, milk beverages, lactic acid bacteria beverages, carbonated beverages, fruit juices, vegetable juices, vegetable or fruit beverages, powdered beverages, jelly drinks, coffee beverages, tea beverages, green tea beverages, sport drinks, nutritional beverages, energy drinks, non-alcoholic beverages, and alcoholic beverages. The above-described foods for sick people can be provided for patients who require improvement of red blood cell function.

Further, when the agent for improving red blood cell function of the present invention is used in the field of food or beverage products, the agent for improving red blood cell function of the present invention, either alone or in combination with other components, can be provided as a food additive for use in improving red blood cell function.

When the agent for improving red blood cell function of the present invention is used for a food or beverage product, the amount of the agent for improving red blood cell function incorporated into the food or beverage product may be set as appropriate within a range where the above-described dose can be satisfied, in accordance with the type, form, and the like of the food or beverage product. For example, the amount of cucurbitaxanthin A and/or a derivative thereof may be in the range from 0.000001 to 20 mass %, preferably from 0.000002 to 18 mass %, and even more preferably from 0.000005 to 15 mass %.

When the agent for improving red blood cell function of the present invention is used in the field of pharmaceutical preparations, cucurbitaxanthin A and/or a derivative thereof, either alone or in combination with other pharmacological components, pharmacologically acceptable bases or additives, and the like, may be prepared into a desired dosage form, and provided as a pharmaceutical preparation for use in improving red blood cell function. Specific examples of such forms of pharmaceutical preparations include, but are not particularly limited to, pharmaceutical preparations for oral administration such as tablets, granules, powders, capsules, soft capsules, and syrups; and pharmaceutical preparations for systemic administration such as injections and infusions. Among the above, a preferred example is a pharmaceutical preparation for oral administration.

When the agent for improving red blood cell function of the present invention is used for a pharmaceutical preparation, the amount of the agent for improving red blood cell function incorporated into the pharmaceutical preparation may be set as appropriate within a range where the above-described dose can be satisfied, in accordance with the type, dosage form, and the like of the pharmaceutical preparation. For example, the amount of cucurbitaxanthin A and/or a derivative thereof may be in the range from 0.00001 to 80 mass %, preferably from 0.00002 to 75 mass %, and even more preferably from 0.0005 to 70 mass %.

### EXAMPLES

The present invention will be specifically described hereinafter with examples; however, the present invention should not be construed as being limited to these examples.

### Example 1

In this test, paprika carotenoids rich in carotenoids having 3,6-epoxy end groups or 5-membered ring end groups were orally administered to an adult male, and cucurbitaxanthin A, capsanthin, capsorubin, and capsanthin 3,6-epoxide were evaluated for their ability to migrate into red blood cells. A specific testing method was as described below.

Initially, 15 g of a paprika pigment emulsified preparation (PapriX; total amount of carotenoids: 10 mg/g) from Glico Nutrition Co., Ltd., 30 g of dextrin, 70 g of sugar, and 3 g of citric acid were dissolved in 1 L of water to prepare a test beverage containing the paprika carotenoids. The paprika pigment emulsified preparation contained 0.053 mass % of cucurbitaxanthin A, 0.40 mass % of capsanthin, 0.021 mass % of capsorubin, and 0.034 mass % of capsanthin 3,6-epoxide, and the test beverage contained 0.00079 mass % of cucurbitaxanthin A, 0.0060 mass % of capsanthin, 0.00031 mass % of capsorubin, and 0.00051 mass % of capsanthin 3,6-epoxide.

A healthy male in his forties continuously ingested 100 mL (200 ml/day) of the test beverage each in the morning and evening for four weeks. Before the beginning of the ingestion and after 4 weeks from the ingestion, blood was collected to obtain blood samples. Red blood cells were separated from the obtained blood samples, and the carotenoids (capsanthin 3,6-epoxide, cucurbitaxanthin A, capsanthin, and capsorubin) present in red blood cells were quantified by HPLC.

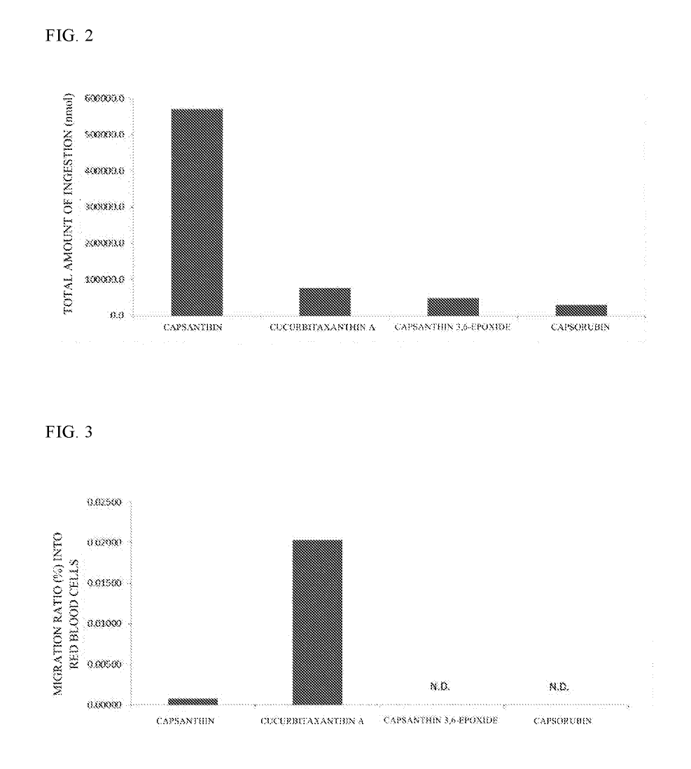

FIG. 2 shows the total amount of ingestion of each of the carotenoids through the ingestion of the test beverage for 4 weeks, and FIG. 3 shows the proportion of the amount of each of the carotenoids present in the red blood cells, relative to the total amount of ingestion of each of the carotenoids (migration ratio into red blood cells; %). The results confirmed that cucurbitaxanthin A has a markedly high ability to migrate into red blood cells. On the other hand, the carotenoids having 3,6-epoxy end groups or 5-membered ring end groups other than cucurbitaxanthin A (capsanthin, capsorubin, and capsanthin 3,6-epoxide) showed little migration into red blood cells.

Cucurbitaxanthin A is a carotenoid having 3,6-epoxy end groups, which has high antioxidant action, and has been believed from the conventional art to lack the ability to migrate into red blood cells. The results obtained in this test, however, revealed that cucurbitaxanthin A in fact has a markedly high ability to migrate into red blood cells, and can effectively protect red blood cells against oxidative stress, and thus, is effective for improving the function of red blood cells.

1. A method of improving red blood cell function comprising the step of administering, to an individual in need of improvement of red blood cell function, cucurbitaxanthin A and/or a derivative thereof in an amount effective for improving red blood cell function.

2. The method of improving red blood cell function according to claim 1, wherein cucurbitaxanthin A and/or a derivative thereof is derived from paprika.

3. The method of improving red blood cell function according to claim 1, wherein cucurbitaxanthin A and/or a derivative thereof is administered in a form of a food or beverage product.

4. The method of improving red blood cell function according to claim 1, wherein cucurbitaxanthin A and/or a derivative thereof is administered in a form of a pharmaceutical preparation.

5. The method of improving red blood cell function according to claim 1, wherein red blood cells are protected against oxidative stress by improvement of red blood cell function.

6. The method of improving red blood cell function according to claim 1, wherein oxygen-carrying capacity of red blood cells is improved by improvement of red blood cell function.

7. The method of improving red blood cell function according to claim 1, wherein oxidative damage to red blood cells is suppressed by improvement of red blood cell function.

8. The method of improving red blood cell function according to claim 1, wherein physical function is improved by improvement of red blood cell function.

9. The method of improving red blood cell function according to claim 1, wherein physical function is recovered during or after exercise by improvement of red blood cell function.

10. The method of improving red blood cell function according to claim 1, wherein cardiopulmonary function is improved by improvement of red blood cell function.

11. The method of improving red blood cell function according to claim 1, wherein brain function is maintained or improved by improvement of red blood cell function.

12. The method of improving red blood cell function according to claim 1, wherein dementia is prevented or treated by improvement of red blood cell function.
